(19) United States
(12) Patent Application Publication
(10) Pub. No.: US 2026/0263142 A1
(43) Pub. Date: Sep. 10, 2026

(54) ELECTROSURGICAL PENCIL WITH SMOKE EVACUATION AND ELECTRODE VISUALIZATION

(71) Applicant: Covidien LP, Mansfield, MA (US)

(72) Inventors: Jason T. SANDERS, Longmont, CO (US); Jenna DANCY, Longmont, CO (US); James H. BODMER, Arvada, CO (US); Tony G. MOUA, Thornton, CO (US)

(21) Appl. No.: 19/165,619

(22) PCT Filed: Mar. 14, 2024
(86) PCT No.: PCT/IB2024/052468

§ 371 Date: Sep. 16, 2025

Related U.S. Application Data
(60) Provisional application No. 63/456,060, filed on Mar. 31, 2023.

(51) Int. Cl. A61B 18/14; A61B 34/00; A61B 34/37; A61M 1/00

(52) U.S. Cl. CPC A61B 18/1402; A61B 34/37; A61B 34/76; A61B 2218/008; A61M 1/87

(57) ABSTRACT

An electrosurgical pencil includes a handle housing defining a longitudinal axis. A nozzle is slidably received within a fluid lumen defined by the handle housing and is configured to move along the longitudinal axis relative to the handle housing between a retracted position and an extended position. The nozzle defines a fluid lumen in fluid communication with the fluid lumen defined by the handle housing for evacuating fluid from a surgical site. An electrode is at least partially disposed within the fluid lumen defined by the nozzle and is configured to deliver electrosurgical energy to tissue. A twist lock is rotatably coupled to a distal end portion of the handle housing and is configured to rotate about the longitudinal axis between a locked position to prevent movement of the nozzle along the longitudinal axis and an unlocked position to permit movement of the nozzle along the longitudinal axis.

This Application claims priority from U.S. Provisional Patent Application 63/456,060, filed 31 Mar. 2023, the entire content of which is incorporated herein by reference.

### FIELD

The disclosure relates to electrosurgical devices. More specifically, the disclosure relates to handheld electrosurgical pencils with smoke evacuation and electrode visualization.

### BACKGROUND

Electrosurgical (ES) pencils are used in surgery, typically for cutting tissue and/or for coagulating blood vessels. An ES pencil usually includes a handpiece into which electrodes of various shapes and sizes may be placed. The ES pencil is coupled to an ES generator, such as Medtronic's Valleylab™ FX8 or FT10 generator, which supplies the electrode with a high frequency, typically radio frequency (RF) alternating current. The ES generator may supply various waveforms suitable for achieving various surgical effects, such as cutting, coagulating, blending, spraying, fulgurating, and the like.

While using an ES pencil, surgical smoke is often generated. An effective way to evacuate surgical smoke and/or other fluids from a surgical site is to use an ES pencil with an integrated smoke evacuation nozzle in conjunction with a suction device and an ultra-low penetration air (ULPA) filter. Conventional ES pencils rely on a smoke evacuation nozzle situated near the pencil's electrode, which draws smoke into and through the pencil's body, through a long flexible hose, and finally into a powered suction device. Smoke evacuation nozzles are available either as an integrated part of the ES pencil or as a separate component attached to the ES pencil. However, conventional ES pencils with smoke evacuation nozzles are not constructed to optimize smoke capture and electrode visualization.

### SUMMARY

Provided in accordance with aspects of the present disclosure is an electrosurgical pencil. The electrosurgical pencil includes a handle housing defining a longitudinal axis. The electrosurgical pencil also includes a nozzle slidably received within a fluid lumen defined by the handle housing. The nozzle is configured to move along the longitudinal axis relative to the handle housing between a retracted position and an extended position. The nozzle defines a fluid lumen in fluid communication with the fluid lumen defined by the handle housing for evacuating fluid from a surgical site. An electrode is at least partially disposed within the fluid lumen defined by the nozzle and is configured to deliver electrosurgical energy to tissue. The electrode is operably coupled to the nozzle such that movement of the nozzle along the longitudinal lumen causes corresponding movement of the electrode along the longitudinal axis. A twist lock is rotatably coupled to a distal end portion of the handle housing and is configured to rotate about the longitudinal axis between a locked position to prevent movement of the nozzle and the electrode along the longitudinal axis, and an unlocked position to permit movement of the nozzle and the electrode along the longitudinal axis.

In an aspect of the present disclosure, a distance between a distal end of the electrode and a distal end of the nozzle remains constant during movement of the nozzle and the electrode along the longitudinal axis.

In another aspect of the present disclosure, rotation of the twist lock to the locked position biases the distal end portion of the handle housing into engagement with the nozzle to prevent movement of the nozzle along the longitudinal axis.

In another aspect of the present disclosure, the nozzle includes a plurality of teeth configured to be engaged by one or more lockout teeth disposed at the distal end portion of the handle housing.

In still another aspect of the present disclosure, the plurality of teeth of the nozzle are passively engaged by the one or more lockout teeth when the twist lock is in the unlocked position such that the nozzle and the electrode are permitted to move along the longitudinal axis.

In yet another aspect of the present disclosure, rotation of the twist lock to the locked position biases the one or more locking teeth into locking engagement with the plurality of teeth of the nozzle to prevent movement of the nozzle and the electrode along the longitudinal axis.

In another aspect of the present disclosure, an inner surface of the twist lock defines a retention recess configured to receive a portion of the distal end portion of the handle housing upon rotation of the twist lock to the locked position.

In another aspect of the present disclosure, receipt of the portion of the distal end portion of the handle housing within the retention recess generates tactile feedback to indicate that the twist lock is in the locked position.

In still another aspect of the present disclosure, the distal end portion of the handle housing is disposed within a lumen defined by the twist lock, and the nozzle is configured to move within the lumen defined by the twist lock along the longitudinal axis.

In yet another aspect of the present disclosure, the electrosurgical pencil also includes an electrical unit disposed within the handle housing and configured to enable delivery of electrosurgical energy to the electrode. At least a portion of the electrical unit is configured to move along the longitudinal axis in response to movement of the nozzle and the electrode along the longitudinal axis.

In still yet another aspect of the present disclosure, a lockout stop extends from an outer surface of the distal end portion of the handle housing and is configured to engage a recess defined within an inner surface of the twist lock upon rotation of the twist lock to the locked position to prevent over-rotation of the twist lock.

In another aspect of the present disclosure, a stop structure extends from an outer surface of the nozzle. In aspects, the stop structure is configured to engage a distal abutment surface disposed within the fluid lumen defined by the handle housing upon movement of the nozzle to the extended position to prevent over-extension of the nozzle. In aspects, the stop structure is configured to engage a proximal abutment surface disposed within the fluid lumen defined by the handle housing upon movement of the nozzle to the retracted position to prevent over-retraction of the nozzle.

In another aspect of the present disclosure, the electrode has a proximal portion coupled to the handle housing and a distal portion extending through the fluid lumen defined by the nozzle such that at least a portion of the electrode extends distally from a distal end of the nozzle.

In yet another aspect of the present disclosure, the electrode is axially offset from the longitudinal axis defined by the handle housing.

Another electrosurgical pencil provided in accordance with the present disclosure includes a handle housing defining a longitudinal axis. A nozzle is slidably received within a fluid lumen defined by the handle housing and is configured to move along the longitudinal axis relative to the handle housing between a retracted position and an extended position. The nozzle defines a fluid lumen in fluid communication with the fluid lumen defined by the handle housing for evacuating fluid from a surgical site. An electrode is at least partially disposed within the fluid lumen defined by the nozzle and is configured to deliver electrosurgical energy to tissue. A twist lock is rotatably coupled to a distal end portion of the handle housing and is configured to rotate about the longitudinal axis between a locked position to prevent movement of the nozzle along the longitudinal axis and an unlocked position to permit movement of the nozzle along the longitudinal axis.

In an aspect of the present disclosure, the electrode is operably coupled to the nozzle such that movement of the nozzle along the longitudinal lumen causes corresponding movement of the electrode along the longitudinal axis.

In another aspect of the present disclosure, the nozzle includes a plurality of teeth configured to be engaged by one or more lockout teeth disposed at the distal end portion of the handle housing.

In still another aspect of the present disclosure, the plurality of teeth of the nozzle are passively engaged by the one or more lockout teeth when the twist lock is in the unlocked position such that the nozzle and the electrode are permitted to move along the longitudinal axis.

In yet another aspect of the present disclosure, rotation of the twist lock to the locked position biases the one or more locking teeth into locking engagement with the plurality of teeth to prevent movement of the nozzle and the electrode along the longitudinal axis.

Another electrosurgical pencil provided in accordance with the present disclosure includes a handle housing defining a longitudinal axis and includes a flexible portion disposed at a distal end portion of the handle housing. A nozzle is slidably received within a fluid lumen defined by the handle housing and is configured to move along the longitudinal axis relative to the handle housing between a retracted position and an extended position. The nozzle defines a fluid lumen in fluid communication with the fluid lumen defined by the handle housing for evacuating fluid from a surgical site. An electrode is at least partially disposed within the fluid lumen defined by the nozzle and is configured to deliver electrosurgical energy to tissue. A twist lock is rotatably coupled to the distal end portion of the handle housing and is configured to rotate about the longitudinal axis between a locked position wherein the twist lock biases the flexible portion into locking engagement with the nozzle to prevent movement of the nozzle along the longitudinal axis and an unlocked position wherein the flexible portion passively engages the nozzle to permit movement of the nozzle along the longitudinal axis.

### BRIEF DESCRIPTION OF THE DRAWINGS

Aspects of the disclosure are described herein with reference to the accompanying drawings, wherein:

FIG. 1 is a perspective view of a surgical smoke evacuation system, in accordance with aspects of the disclosure;

FIG. 2 is a side perspective view of an electrosurgical (ES) pencil of the surgical smoke evacuation system of FIG. 1, in accordance with aspects of the disclosure;

FIG. 3 is an exploded perspective view of the ES pencil of FIG. 2, in accordance with aspects of the disclosure;

FIGS. 4A and 4B are side cross-sectional views of the ES pencil of FIG. 2, in accordance with aspects of the disclosure;

FIG. 5 is a side perspective view of an electrical unit coupled to an electrode of the ES pencil of FIG. 2, in accordance with aspects of the disclosure;

FIG. 6 is a side perspective view of a portion of the ES pencil of FIG. 2 with portions removed, in accordance with aspects of the disclosure;

FIG. 7 is a side perspective view of a portion of the ES pencil of FIG. 2 with portions removed, in accordance with aspects of the disclosure;

FIG. 8 is a front perspective view of a portion of the ES pencil of FIG. 2 with portions removed, in accordance with aspects of the disclosure;

FIGS. 9A and 9B are rear cross-sectional views of a portion of the ES pencil of FIG. 2, in accordance with aspects of the disclosure;

FIG. 10A is a front perspective view of a portion of the ES pencil of FIG. 2 with portions removed, in accordance with aspects of the disclosure;

FIG. 10B is a side perspective view of a portion of the ES pencil of FIG. 2 with portions removed, in accordance with aspects of the disclosure;

FIG. 11A is a side perspective cross-sectional view of the portion of the ES pencil of FIG. 2, in accordance with aspects of the disclosure;

FIG. 11B is a side cross-sectional view of a portion of the ES pencil of FIG. 2, in accordance with aspects of the disclosure;

FIG. 12A is a rear perspective cross-sectional view of a portion of the ES pencil of FIG. 2, in accordance with aspects of the disclosure;

FIG. 12B is a rear perspective cross-sectional view of a portion of the ES pencil of FIG. 2, in accordance with aspects of the disclosure;

FIG. 12C is a rear cross-sectional view of a portion of the ES pencil of FIG. 2, in accordance with aspects of the disclosure; and

FIG. 13 is a schematic illustration of an exemplary robotic surgical system configured for use with the present disclosure.

### DETAILED DESCRIPTION

Embodiments of the disclosure are now described in detail with reference to the drawings in which like reference numerals designate identical or corresponding elements in each of the drawings. The aspects may be combined in any manner consistent with the functionality of the apparatus and/or method disclosed herein. As used herein, the term “clinician” refers to a doctor, a surgeon, a nurse, or any other care provider and may include support personnel. Throughout this description, the term “proximal” will refer to the portion of the device or component thereof that is closer to the clinician and the term “distal” will refer to the portion of the device or component thereof that is farther from the clinician. As used herein, the term “exemplary” does not necessarily mean “preferred” and may simply refer to an example unless the context clearly indicates otherwise.

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent.

Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the disclosure in unnecessary detail.

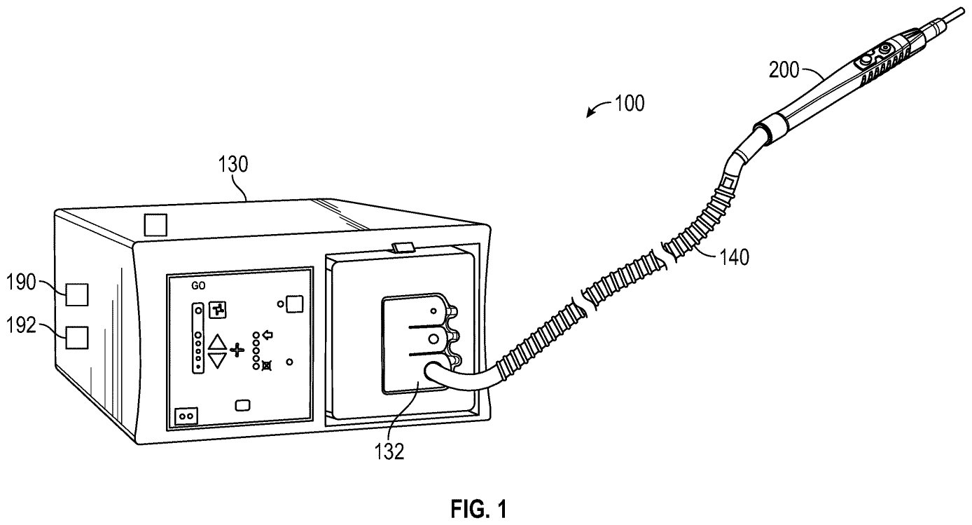

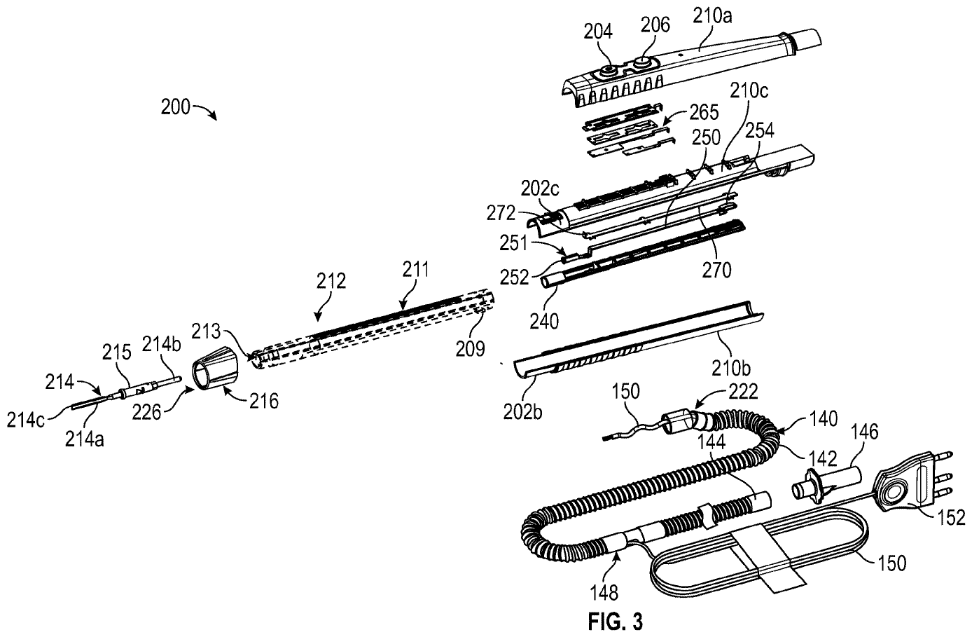

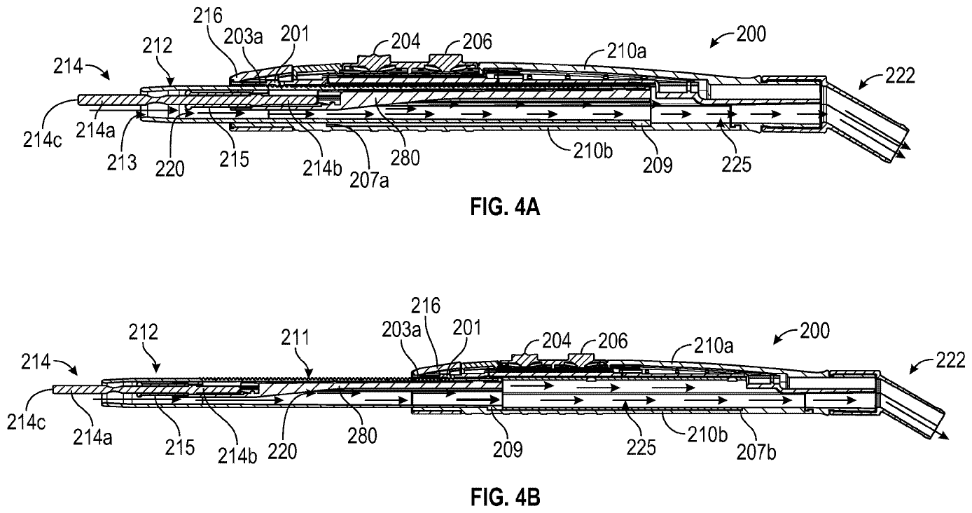

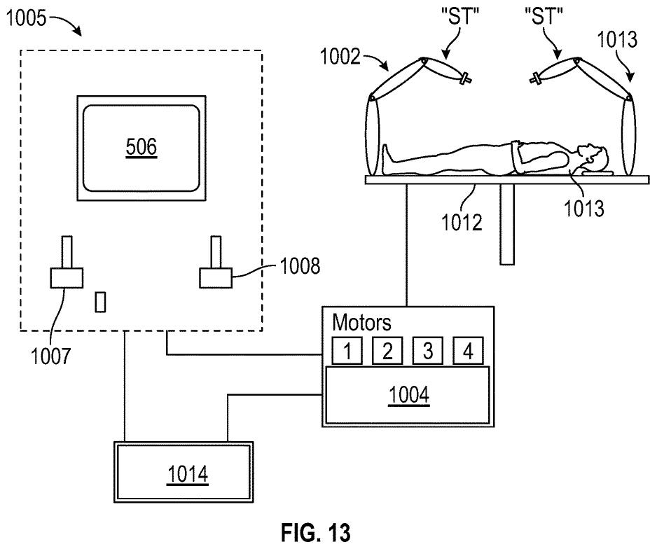

The present disclosure relates to an ES pencil that is constructed to effectively accomplish smoke capture and electrode visualization during operation of the ES pencil to treat tissue and evacuate fluid (e.g., surgical smoke, debris, gaseous byproducts, etc.) from the surgical site. With reference to FIGS. 1-3, a surgical smoke evacuation system 100 in accordance with aspects of the present disclosure is shown. The surgical smoke evacuation system 100 generally includes a smoke evacuator 130, an ES pencil 200 having a handle housing 210, and tubing 140 connecting the ES pencil 200 to the smoke evacuator 130. The handle housing 210 may be configured as a handle configured to be gripped by a clinician, although non-handle configurations are also contemplated, e.g., for mounting the ES pencil 200 and/or attaching the ES pencil 200 to a surgical robot arm (see FIG. 13). The smoke evacuator 130 may include one or more fans and/or pumps configured to create negative pressure (e.g., a vacuum force) for removing surgical smoke and/or debris from a surgical site. A connector 222 (e.g., swivel connector) at a distal end of the tubing 140 couples the handle housing 210 to the tubing 140. A proximal end 144 of the tubing 140 is configured for connection to an inlet port 132 of the smoke evacuator 130. Optionally, the proximal end 144 of the tubing 140 may couple to an adapter 146 (FIG. 3) configured to accommodate connection of the tubing 140 to various sized inlet ports of the smoke evacuator 130. The tubing 140 is in fluid communication with a fluid lumen 225 (FIGS. 4A and 4B), e.g., a smoke lumen, defined through the handle housing 210 of the ES pencil 200. The fluid lumen 225 defined through the handle housing 210 may be defined by the handle housing 210 itself or, in some aspects, may be a separate tube or luminal structure disposed within the handle housing 210 or coupled to an exterior of the handle housing 210. The tubing 140 may be corrugated by including a spiral spine 142 disposed on an outer surface of the tubing 140. The corrugated structure of the tubing 140 minimizes kinking and provides increased flexibility to the tubing 140. As shown in FIG. 3, the tubing 140 may also include an opening 148 at any point along its length for passage of an electrosurgical cable 150 into a lumen defined within the tubing 140. The cable 150 includes a proximal connector 152 (FIG. 3) configured for connection to an electrosurgical generator (not shown). A lumen defined through the connector 222 is in fluid communication with the lumen defined through the tubing 140 and the fluid lumen 225 defined through the handle housing 210. Thus, the fluid lumen 225 defined through the handle housing 210 is configured to be in fluid communication with the inlet port 132 of the smoke evacuator 130 via the connector 222 and the tubing 140 for evacuating fluid (e.g., surgical smoke) from a surgical site.

The smoke evacuator 130 also includes a processor 190 and a memory 192. Instructions may be executed by the processor 190, which may include one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements. It is contemplated that the processor 190 and memory 192 may be located in the smoke evacuator 130, the ES pencil 200, and/or in a remote computer system.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

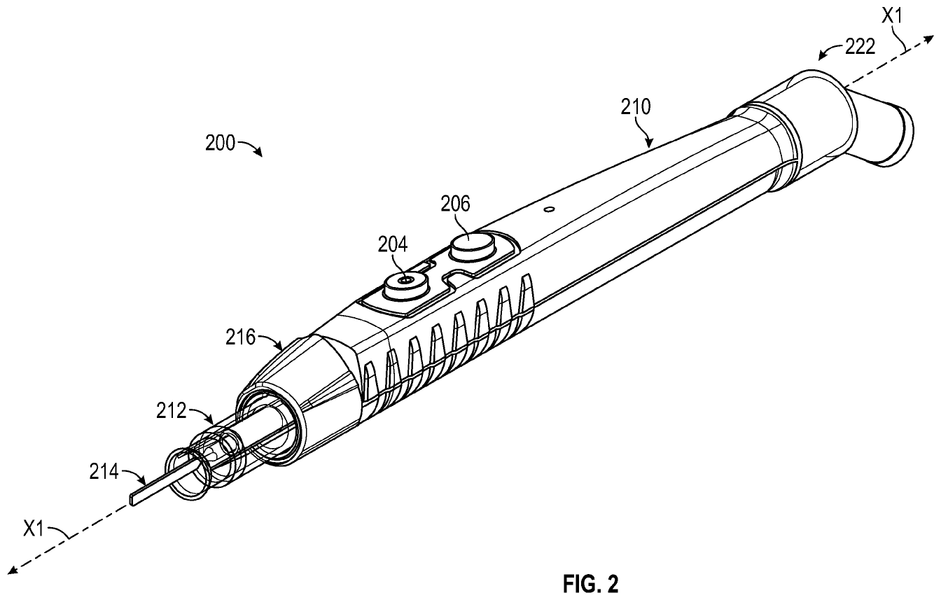

Referring to FIGS. 2-4B, the ES pencil 200 generally includes a handle housing 210 defining a longitudinal axis X1, an electrical unit 260, a nozzle 212 extending distally from the handle housing 210, and an electrode 214 extending distally from the handle housing 210 and at least partially through a fluid lumen 220 (e.g., smoke lumen) defined through the nozzle 212. The nozzle 212 is slidably received within the fluid lumen 225 defined through the handle housing 210 and is movable relative to the handle housing 210 to a plurality of different positions along the longitudinal axis X1. A plurality of teeth 211 are formed on an outer surface of the nozzle 212 and along at least a portion of a longitudinal length of the nozzle 212. The electrode 214 is operably coupled to the nozzle 212 and is configured to move in unison with the nozzle 212 along the longitudinal axis X1 such that the distance between the distal tip 214c of the electrode 214 and a distal end 212c of the nozzle 212 remains constant during movement of the nozzle 212 and the electrode 214 along the longitudinal axis X1. Moving the nozzle 212 and/or the electrode 214 along the longitudinal axis X1 serves to adjust a length of the nozzle 212 that extends distally from the handle housing 210 and to adjust a distance between a distal tip 214c of the electrode 214 and a distal end of the housing 210. FIG. 4A shows the nozzle 212 and the electrode 214 in a fully retracted position. FIG. 4B shows the nozzle 212 and the electrode 214 in a fully extended position. It should be understood that the nozzle 212 and the electrode 214 may be extended to any one of a plurality of extended positions between the fully extended position shown in FIG. 4B and the fully retracted position shown in FIG. 4A. A proximal stop structure 209 disposed on a bottom of the nozzle 212 serves to prevent over-retraction of the nozzle 212 by abutting a proximal abutment surface 207b defined by the bottom housing portion 210b upon proximal movement of the nozzle 212 to the fully retracted position, as shown in FIG. 4A. Likewise, the proximal stop structure 209 serves to prevent over-extension of the nozzle 212 by abutting a distal abutment surface 207a defined by the bottom housing portion 210b upon distal movement of the nozzle to the fully extended position, as shown in FIG. 4B.

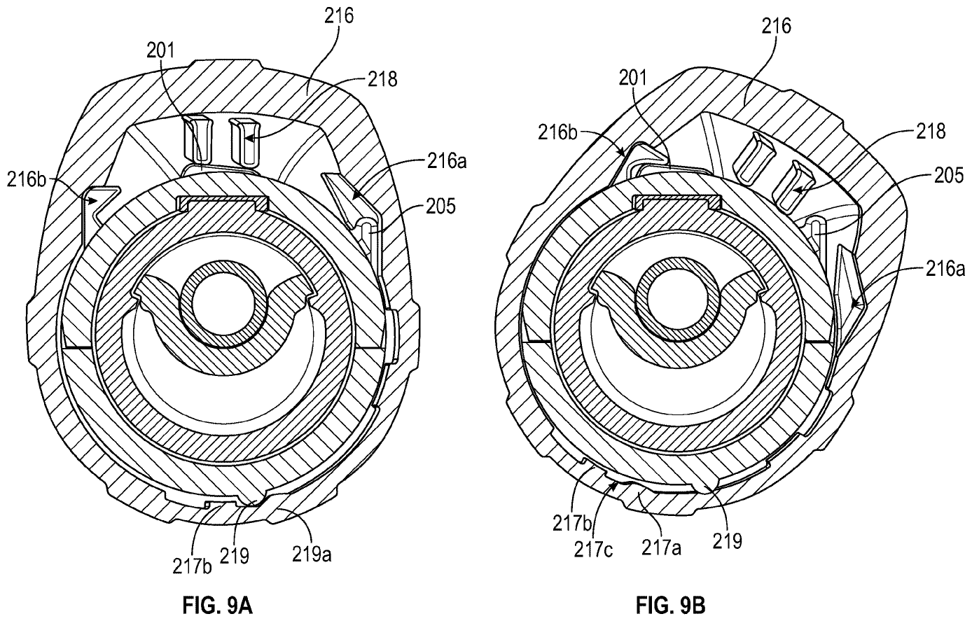

The ES pencil 200 also includes a twist lock 216 coupled to the handle housing 210. As detailed below, the twist lock 216 is configured to rotate about the longitudinal axis X1 and relative to the handle housing 210 to releasably lock the nozzle 212 in any one of a plurality of axial positions along the longitudinal axis X1. When the twist lock 216 is rotated relative to the handle housing 210 into a locked position (FIG. 9A), the nozzle 212 and the electrode 214 are prevented from moving along the longitudinal axis X1. When the twist lock 216 is rotated relative to the handle housing 210 into an unlocked position (FIG. 9B), the nozzle 212 and the electrode 214 are freely movable along the longitudinal axis X1 to enable the clinician to adjust the axial position of the nozzle 212 and the electrode 214 along the longitudinal axis X1. In aspects of the present disclosure, the twist lock 216 may be rotated into any one of a plurality of unlocked positions in which the twist lock 216 is not in the locked position shown in FIG. 9A. Once the nozzle 212 and the electrode 214 are moved to a desired axial position along the longitudinal axis X1, the twist lock 216 may be rotated relative to the handle housing 210 into the locked position to releasably lock the nozzle 212 and the electrode 214 at the desired axial position along the longitudinal axis X1.

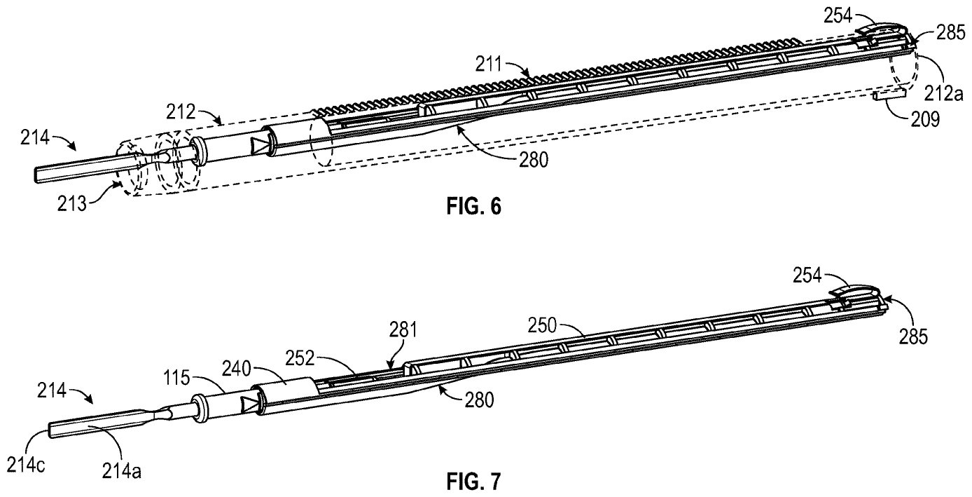

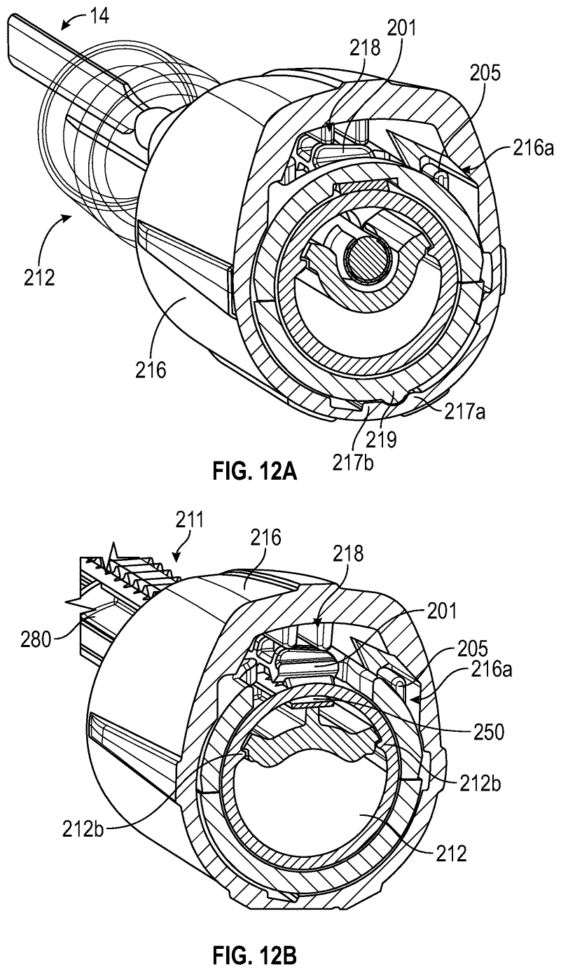

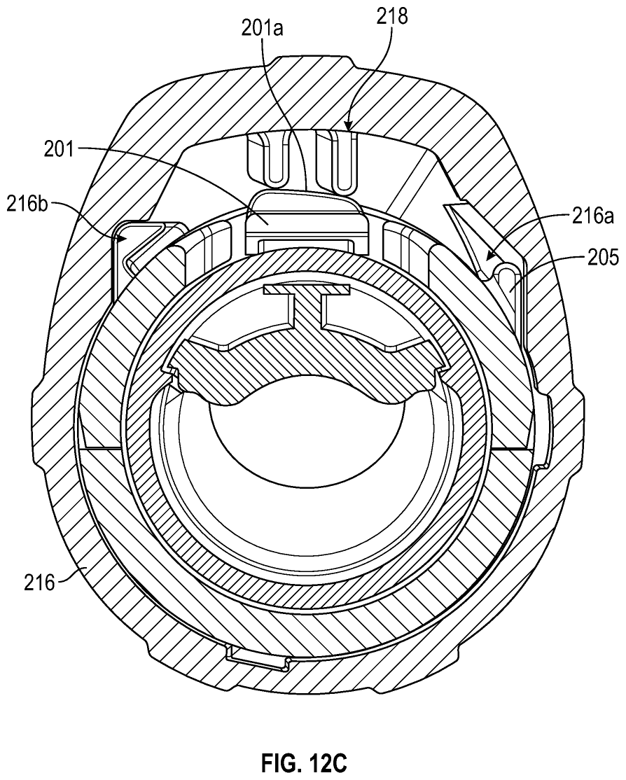

The handle housing 210 of the ES pencil 200 may be formed from a thermoplastic material and includes a top housing portion 210a, a bottom housing portion 210b, and a midframe housing portion 210c disposed between the top and bottom housing portions 210a, 210b. The top and bottom housing portions 210a, 210b are secured to each other using any suitable method (e.g., ultrasonic welding) to secure and house the midframe housing portion 210c and other internal components of the ES pencil 200. The bottom housing portion 210b and the midframe housing portion 210c include respective recessed distal end portions 202b, 202c (hereinafter referred to in combination as the recessed distal end portion 202) that terminate at respective distal ends 229b, 229c (hereinafter referred to in combination as the distal end 229). A clip carrier 280 is operably coupled to the electrical unit 260 and slidably disposed within the handle housing 210 between the bottom housing portion 210b and the midframe housing portion 210c. The clip carrier 280 is disposed within the fluid lumen 220 defined through the nozzle 212 and is seated on a pair of opposing longitudinal protrusions 212b (FIG. 12B) extending along at least a portion of an inner surface of the nozzle 212. A proximal end portion 285 of the clip carrier 280 is configured to attach to a proximal end rim 212a (FIG. 6) of the nozzle 212 such that the clip carrier 280 is configured to move along the longitudinal axis in unison with the nozzle 212. As detailed below, the clip carrier 280 is operably coupled to the electrode 214 to facilitate simultaneous movement of the nozzle 212 and the electrode 214 along the longitudinal axis X1.

The nozzle 212 extends distally from the handle housing 210 and is configured for suctioning fluid (e.g., surgical smoke, debris, gaseous byproducts, etc.) from a surgical site. The nozzle 212 is slidably disposed within the handle housing 210 between the bottom housing portion 210b and the midframe housing portion 210c. The fluid lumen 220 defined through the nozzle 212 is configured for suctioning fluid (e.g., surgical smoke, debris, gaseous byproducts, etc.) from a surgical site through a distal opening 213 of the nozzle 212. The fluid lumen 220 defined through the nozzle 212 is in fluid communication with the fluid lumen 225 defined through the handle housing 210 is placed in fluid communication with the fluid lumen 220 defined through the nozzle 212 to enable fluid (e.g., smoke) suctioned through the nozzle 212 to be evacuated through the handle housing 210 and the tubing 140 via operation of the smoke evacuator 130. Depicted in FIGS. 4A and 4B are arrows to illustrate the airflow path through which fluid (e.g., smoke) is evacuated through the fluid lumen 220 defined through the nozzle 212 and the fluid lumen 225 defined through the handle housing 210.

In aspects of this disclosure, at least a portion of the nozzle 212 may be a transparent, substantially transparent, or translucent material configured to facilitate visual acuity in the surgical field. For example, the nozzle 212 may be formed from a clear polycarbonate resin. Other resin materials from which to form the nozzle 212 are contemplated such as, for example, polymethylmethacrylate or acrylic (PMMA), polymethylmethyacrylimide (PMMI), silicon-based resins, or the like. In aspects of this disclosure, the nozzle 212 may be at least partially formed from a radiopaque material such as, for example, a thermoplastic polyurethane (TPU) material so that the nozzle 212 appears opaque under medical imaging modalities that use radiation such as X-rays for example.

The electrode 214 includes a distal portion 214a having a tissue treatment portion (e.g., a blade (as shown), a hook, a needle, etc.) and a proximal portion 214b disposed within the fluid lumen 220 of the nozzle 212. The electrode 214 is offset from the longitudinal axis X1 towards the top of the ES pencil 200 to enhance electrode visibility and to maximize the area of fluid communication between the lumen 220 of the nozzle 212 and the fluid lumen 225 of the handle housing 210 for improving smoke evacuation performance. In this instance, the top of the ES pencil 200 is considered to be a portion of the ES pencil 200 that favors the top housing portion 210a. The electrode 214 is removably received through a collet 215 that is, in turn, supported through a receptacle 240 formed within the clip carrier 280 (FIG. 3). In aspects, the electrode 214 is removable from the collet 215 such that the electrode 214 may be replaced by a new electrode and/or by an electrode having a different shape, size, and/or configuration depending on the needs of the clinician for a given procedure. In aspects of the present disclosure, the nozzle 212 may also be removable from the handle housing 210 such that the nozzle 212 may be replaced by a new nozzle and/or by a nozzle having a different shape, size, and/or configuration. For example, a nozzle may be replaced with a different size nozzle depending on the size of the electrode being used.

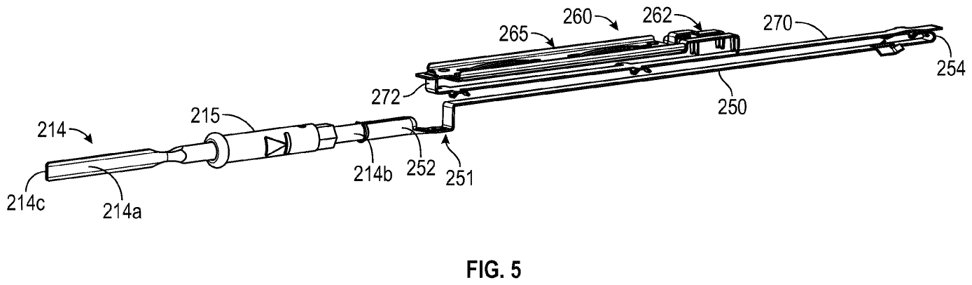

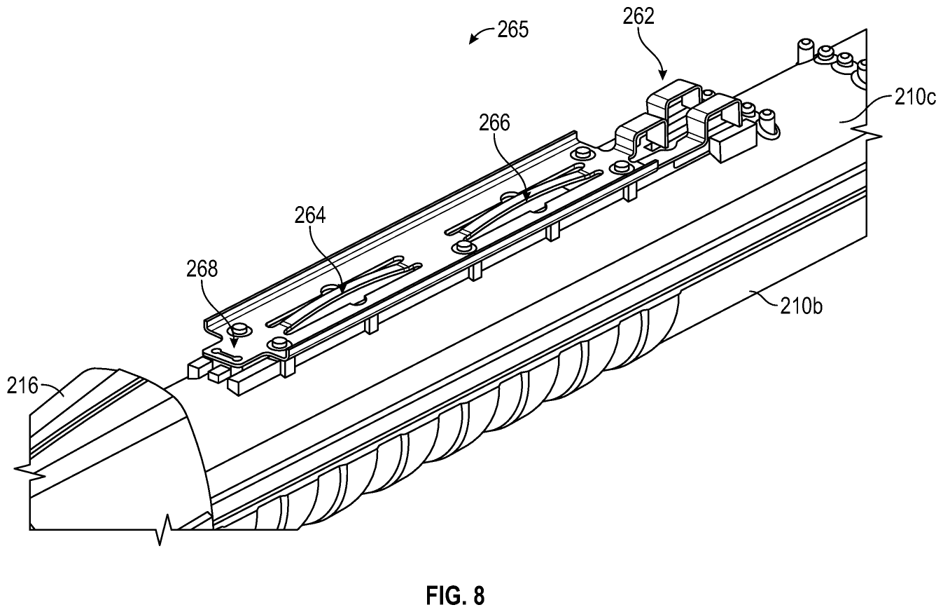

Referring now to FIGS. 3 and 5-8, the electrical unit 260 is configured to facilitate electrical communication between the electrode 214 and an electrosurgical generator (not shown). The electrical unit 260 generally includes a switch assembly 265, an electrically conductive power transfer bar 270, and an electrically conductive clip 280 disposed at least partially within the fluid lumen 220 of the nozzle 212. The proximal portion 214b of the electrode 214 extends proximally from the receptacle 240 of the clip carrier 280 and is received within an electrically conductive electrode clip 252 disposed at a distal end portion 251 of the clip 250. In aspects of the present disclosure, the distal end portion 251 of the clip 250 is axially offset relative to the remainder of the clip 250 such that the distal end portion 251 seats within a longitudinal recessed portion 281 of the clip carrier 280 (FIG. 7) to align the electrode clip 252 with the receptacle 240 for facilitating reception of the proximal portion 214b of the electrode 214 within the electrode clip 252. The clip 250 is coupled to the clip carrier 280 such that the clip 250 and the electrode 214 move along the longitudinal axis X1 in unison with the clip carrier 280. The clip 250 includes a proximal hook portion 254 that hooks around the proximal end rim 212a of the nozzle 212 (FIG. 6) so that the proximal hook portion 254 is exposed from within the fluid lumen 220 of the nozzle 212 and disposed between the power transfer bar 270 and the nozzle 212. The proximal hook portion 254 contacts a bottom side of the power transfer bar 270 and maintains contact with the bottom side of the power transfer bar 270 during movement of the clip carrier 280 along the longitudinal axis X1. In this way, the proximal hook portion 254 serves as a sliding electrical contact with respect to the bottom side of the power transfer bar 270 to maintain electrical communication between the clip 250 and the power transfer bar 254 during movement of the clip 250 (via the clip carrier 280) relative to the power transfer bar 254 along the longitudinal axis X1. The switch assembly 265 is mounted on the midframe housing portion 210c and includes one or more electrical contacts 262 configured to be electromechanically coupled to the cable 150, which interconnects the electrical unit 260 to an electrosurgical generator (not shown). The switch assembly 265 also includes a distal receptacle 268 (FIG. 8) that receives an electrical contact 272 extending from a distal end portion of the power transfer bar 270 to establish electrical communication between the switch assembly 265 and the power transfer bar 270, which in turn establishes electrical communication between the switch assembly 265 and the electrode 214 via contact between the proximal hook portion 254 of the clip 250 and the bottom side of the power transfer bar 270. Since the electrical contact 272 of the power transfer bar 270 is received through the receptacle 268 of the switch assembly 268, the power transfer bar 270 remains stationary as the clip carrier 280 and thus, the clip 250, move along the longitudinal axis X1 and as the proximal hook portion 254 slides along the bottom side of the power transfer bar 270. The switch assembly 265 includes a pair of switches 262, 264 (FIG. 8), which are disposed in vertical registration with a pair of push buttons 204, 206 (FIG. 3), respectively, extending from the top housing portion 210a, thereby allowing for activation of the switches 262, 264 when the corresponding button 204, 206 is pressed. Activation and/or deactivation of pushbutton switches 262, 264 serves to control delivery of electrosurgical energy to the electrode 214. For example, one of the buttons 204, 206 may serve to cause the electrosurgical generator to provide a signal to the electrode 214 for cutting tissue and the other of the buttons 204, 206 may serve to cause the electrosurgical generator to provide a signal to the electrode 214 for coagulating tissue. In aspects of the disclosure, the buttons 204, 206 may be replaced by any suitable actuation mechanism, such as a rocker switch, a pressure sensitive transducer, or a slider configured to be actuated longitudinally (e.g., distally and proximally) along the handle housing 210.

Referring now to FIGS. 9A-12C, the twist lock 216 defines an interior lumen 226 (FIG. 3) configured to receive the recessed distal end portion 202 of the handle housing 210 to rotatably couple the twist lock 216 to the handle housing 210. The twist lock 216 includes one or more lockout retaining arms 218 extending radially inward from an inner surface of the twist lock 216. In aspects of the present disclosure, the one or more lockout retaining arms 218 includes a pair of lockout retaining arms 218 as shown in the example embodiment of FIGS. 9A, 9B, and 12A-12C. Also extending radially inward from an inner surface of the twist lock 216 are a protuberance 217a and a retention stop 217b to form a retention recess 217c therebetween. The retention recess 217c is configured to receive a protuberance 219 extending from an outer surface of the recessed distal end portion 202b of the bottom housing portion 210b upon rotation of the twist lock 216 to a locked position (FIG. 9A).

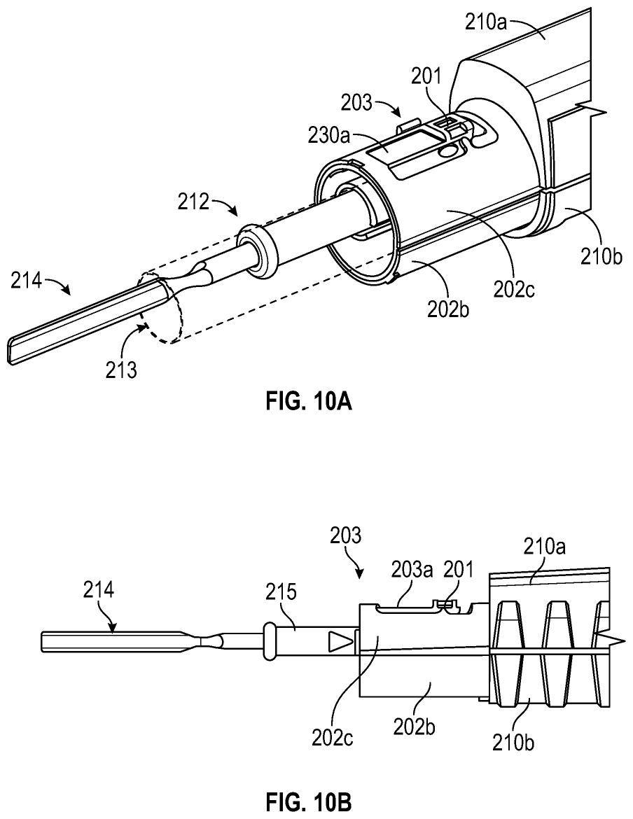

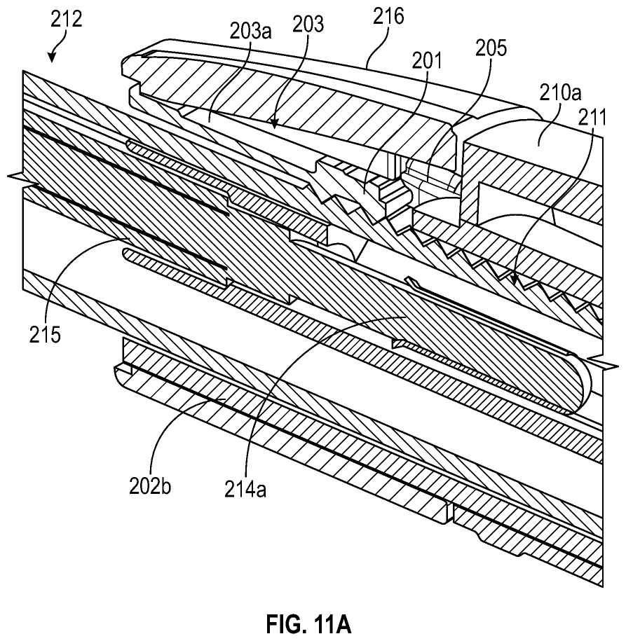

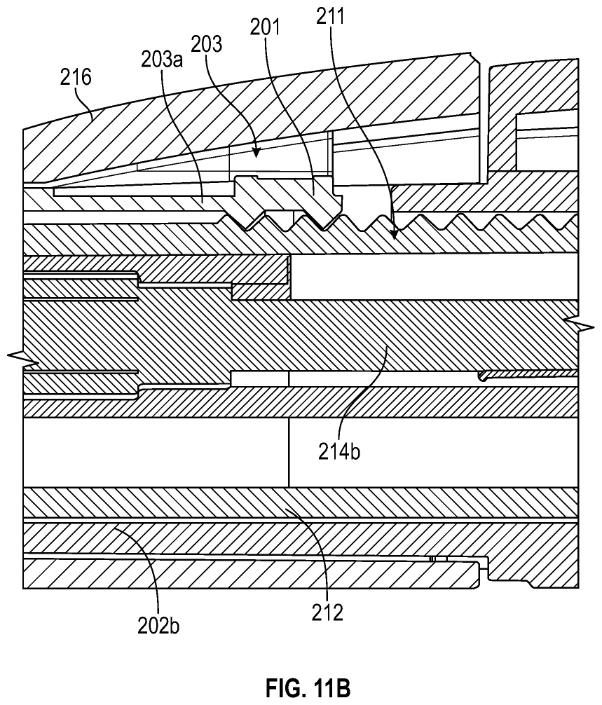

The recessed distal end portion 202c of the midframe housing portion 210c includes a flexible portion 203 (FIGS. 10A and 10B) having a cantilevered flexible beam 203a and one or more lockout teeth 201 (FIGS. 11A and 11B) extending from a free end of the flexible beam 203a. In aspects of the present disclosure, the flexible portion 203 may be molded as part of the recessed distal end portion 202c of the midframe housing portion 210c. The one or more lockout teeth 201 are configured to engage the plurality of teeth 211 of the nozzle 212 (FIGS. 11A and 11B). When the twist lock 216 is rotated (e.g., clockwise as viewed from a distal end of the ES pencil 200) relative to the handle housing 210 toward the locked position (FIG. 9A), the one or more lockout retaining arms 218 are rotated into vertical registration with the one or more lockout teeth 201 (FIG. 12C) such that the one or more lockout retaining arms 218 biases the one or more lockout teeth 201 into locking engagement with the plurality of teeth 211 of the nozzle 212 to prevent longitudinal movement of the nozzle 212 and thus, the electrode 214, along the longitudinal axis X1. Extending outward from an outer surface of the recessed distal end portion 202c of the midframe housing portion 210c is a lockout stop 205 that abuts a recessed portion 216a of an inner surface of the twist lock 216, thereby preventing over-rotation of the twist lock 216 (e.g., clockwise rotation as viewed from a distal end of the ES pencil 200) when the twist lock 216 is already at the locked position (FIG. 9A). To prevent over-rotation of the twist lock 2116 toward an unlocked position (e.g., counter-clockwise as viewed from a distal end of the ES pencil 200), at least a portion of the one or more lockout teeth 201 are received within a recessed portion 216b of an inner surface of the twist lock 216, as shown in FIG. 9B. As shown in FIG. 12C, the one or more lockout teeth 201 may form ramped surface 201a (FIG. 12C) that facilitates sliding of the one or more lockout retaining arms 218 into vertical registration with the one or more lockout teeth 201 as the twist lock 216 is rotated into the locked position (FIG. 9A).

When the twist lock 216 is rotated (e.g., clockwise as viewed from a distal end of the ES pencil 200) relative to the handle housing 210 toward an unlocked position (FIG. 9B), the one or more lockout retaining arms 218 are rotated out of vertical registration with the one or more lockout teeth 201 such that the one or more lockout teeth 201 are no longer biased into locking engagement with the plurality of teeth 211 by the one or more lockout retaining arms 218. When the twist lock 216 is in an unlocked position, the plurality of teeth 211 of the nozzle 212 remain passively engaged with the one or more lockout teeth 201, however, the flexibility of the flexible beam 203a allows for the plurality of teeth 211 to bias the flexible beam 203a and the one or more lockout teeth 201 away from the nozzle 212 as the nozzle 212 moves longitudinally along the longitudinal axis X1. In this way, the one or more lockout teeth 201 do not prevent longitudinal movement of the nozzle 212 and/or corresponding longitudinal movement of the electrode 214 when the twist lock 216 is in an unlocked position (FIG. 9B).

Upon rotation of the twist lock 216 toward the locked position (FIG. 9A), the protuberance 219, which is stationary relative to the twist lock 216, is forced to slide over the protuberance 217a of the twist lock 216 to urge the protuberance 219 into the retention recess 217c. The retention stop 217b cooperates with the protuberance 217a to retain the protuberance 219 within the retention recess 217c. As shown in the example of FIGS. 9A and 9B, the protuberances 217a and 219 may be rounded to facilitate sliding relative to one another during rotation of the twist lock 216, although other geometries of the protuberances 217a and 219 are contemplated by the present disclosure. Urging of the protuberance 219 into the retention recess 217c as described above serves to provide tactile feedback for indicating to the clinician that the twist lock 216 is in the locked position and that the nozzle 212 and the electrode 214 will not inadvertently move along the longitudinal axis X1 during use of the ES pencil 200.

To rotate the twist lock 216 from the locked position (FIG. 9A) toward an unlocked position (FIG. 9B), the clinician simply rotates the twist lock 216 (e.g., counter clock-wise as viewed from a distal end of the ES pencil) to force the protuberance 219 to slide back over the protuberance 217a and out of the retention recess 217c. Urging of the protuberance 219 out of the retention recess 217c serves to provide tactile feedback for indicating to the clinician that the twist lock 216 is released from the locked position and that the nozzle 212 and the electrode 214 may be moved along the longitudinal axis X1.

Turning now to FIG. 13, a robotic surgical system 1000 configured for use in accordance with this disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, the ES pencil 200 of the present disclosure, thus providing any of the above-detailed functionality on a robotic surgical system 1000.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An electrosurgical pencil, comprising:
a handle housing defining a longitudinal axis;
a nozzle slidably received within a fluid lumen defined by the handle housing and configured to move along the longitudinal axis relative to the handle housing between a retracted position and an extended position, the nozzle defining a fluid lumen in fluid communication with the fluid lumen defined by the handle housing for evacuating fluid from a surgical site;
an electrode at least partially disposed within the fluid lumen defined by the nozzle and configured to deliver electrosurgical energy to tissue, the electrode operably coupled to the nozzle such that movement of the nozzle along the longitudinal lumen causes corresponding movement of the electrode along the longitudinal axis; and
a twist lock rotatably coupled to a distal end portion of the handle housing and configured to rotate about the longitudinal axis between a locked position to prevent movement of the nozzle and the electrode along the longitudinal axis and an unlocked position to permit movement of the nozzle and the electrode along the longitudinal axis.

2. The electrosurgical pencil according to claim 1, wherein a distance between a distal end of the electrode and a distal end of the nozzle remains constant during movement of the nozzle and the electrode along the longitudinal axis.

3. The electrosurgical pencil according to claim 1, wherein rotation of the twist lock to the locked position biases the distal end portion of the handle housing into engagement with the nozzle to prevent movement of the nozzle along the longitudinal axis.

4. The electrosurgical pencil according to claim 1, wherein the nozzle includes a plurality of teeth configured to be engaged by one or more lockout teeth disposed at the distal end portion of the handle housing.

5. The electrosurgical pencil according to claim 4, wherein the plurality of teeth of the nozzle are passively engaged by the one or more lockout teeth when the twist lock is in the unlocked position such that the nozzle and the electrode are permitted to move along the longitudinal axis.

6. The electrosurgical pencil according to claim 4, wherein rotation of the twist lock to the locked position biases the one or more locking teeth into locking engagement with the plurality of teeth of the nozzle to prevent movement of the nozzle and the electrode along the longitudinal axis.

7. The electrosurgical pencil according to claim 1, wherein an inner surface of the twist lock defines a retention recess configured to receive a portion of the distal end portion of the handle housing upon rotation of the twist lock to the locked position.

8. The electrosurgical pencil according to claim 7, wherein receipt of the portion of the distal end portion of the handle housing within the retention recess generates tactile feedback to indicate that the twist lock is in the locked position.

9. The electrosurgical pencil according to claim 1, wherein the distal end portion of the handle housing is disposed within a lumen defined by the twist lock, and the nozzle is configured to move within the lumen defined by the twist lock along the longitudinal axis.

10. The electrosurgical pencil according to claim 1, further comprising an electrical unit disposed within the handle housing and configured to enable delivery of electrosurgical energy to the electrode, wherein at least a portion of the electrical unit is configured to move along the longitudinal axis in response to movement of the nozzle and the electrode along the longitudinal axis.

11. The electrosurgical pencil according to claim 1, further comprising a lockout stop extending from an outer surface of the distal end portion of the handle housing, the lockout stop configured to engage a recess defined within an inner surface of the twist lock upon rotation of the twist lock to the locked position to prevent over-rotation of the twist lock.

12. The electrosurgical pencil according to claim 1, further comprising a stop structure extending from an outer surface of the nozzle, wherein the stop structure is configured to:
engage a distal abutment surface disposed within the fluid lumen defined by the handle housing upon movement of the nozzle to the extended position to prevent over-extension of the nozzle; and
engage a proximal abutment surface disposed within the fluid lumen defined by the handle housing upon movement of the nozzle to the retracted position to prevent over-retraction of the nozzle.

13. The electrosurgical pencil according to claim 1, wherein the electrode has a proximal portion coupled to the handle housing and a distal portion extending through the fluid lumen defined by the nozzle such that at least a portion of the electrode extends distally from a distal end of the nozzle.

14. The electrosurgical pencil according to claim 1, wherein the electrode is axially offset from the longitudinal axis defined by the handle housing.

15. An electrosurgical pencil, comprising:
a handle housing defining a longitudinal axis;
a nozzle slidably received within a fluid lumen defined by the handle housing and configured to move along the longitudinal axis relative to the handle housing between a retracted position and an extended position, the nozzle defining a fluid lumen in fluid communication with the fluid lumen defined by the handle housing for evacuating fluid from a surgical site;
an electrode at least partially disposed within the fluid lumen defined by the nozzle and configured to deliver electrosurgical energy to tissue; and
a twist lock rotatably coupled to a distal end portion of the handle housing and configured to rotate about the longitudinal axis between a locked position to prevent movement of the nozzle along the longitudinal axis and an unlocked position to permit movement of the nozzle along the longitudinal axis.

16. The electrosurgical pencil according to claim 15, wherein the electrode is operably coupled to the nozzle such that movement of the nozzle along the longitudinal lumen causes corresponding movement of the electrode along the longitudinal axis.

17. The electrosurgical pencil according to claim 15, wherein the nozzle includes a plurality of teeth configured to be engaged by one or more lockout teeth disposed at the distal end portion of the handle housing.

18. The electrosurgical pencil according to claim 17, wherein the plurality of teeth of the nozzle are passively engaged by the one or more lockout teeth when the twist lock is in the unlocked position such that the nozzle and the electrode are permitted to move along the longitudinal axis.

19. The electrosurgical pencil according to claim 17, wherein rotation of the twist lock to the locked position biases the one or more locking teeth into locking engagement with the plurality of teeth to prevent movement of the nozzle and the electrode along the longitudinal axis.

20. An electrosurgical pencil, comprising:
a handle housing defining a longitudinal axis and including a flexible portion disposed at a distal end portion of the handle housing;
a nozzle slidably received within a fluid lumen defined by the handle housing and configured to move along the longitudinal axis relative to the handle housing between a retracted position and an extended position, the nozzle defining a fluid lumen in fluid communication with the fluid lumen defined by the handle housing for evacuating fluid from a surgical site;
an electrode at least partially disposed within the fluid lumen defined by the nozzle and configured to deliver electrosurgical energy to tissue; and
a twist lock rotatably coupled to the distal end portion of the handle housing and configured to rotate about the longitudinal axis between a locked position wherein the twist lock biases the flexible portion into locking engagement with the nozzle to prevent movement of the nozzle along the longitudinal axis and an unlocked position wherein the flexible portion passively engages the nozzle to permit movement of the nozzle along the longitudinal axis.
